(19) United States
(12) Patent Application Publication
(10) Pub. No.: US 2022/0387661 A1
(43) Pub. Date: Dec. 8, 2022

(54) HOT MELT PRESSURE SENSITIVE ADHESIVE COMPOSITIONS AND ARTICLES INCLUDING THE SAME

(71) Applicant: H.B. Fuller Company, St.Paul, MN (US)

(72) Inventors: Daniel A. Everson, Lakeville, MN (US); Maynard R. Lawrence, Oakedale, MN (US)

(21) Appl. No.: 17/804,478

(22) Filed: May 27, 2022

Related U.S. Application Data
(60) Provisional application No. 63/202,103, filed on May 27, 2021.

(51) Int. Cl. A61L 15/58; A61L 15/24; C09J 153/02

(52) U.S. Cl. CPC A61L 15/58; A61L 15/24; C09J 153/02

(57) ABSTRACT

The HMPSA compositions of this invention include from 10% by weight to 35% by weight of a styrenic block copolymer, from 10% by weight to 60% by weight of a near white rosin ester tackifying agent having a neat Molten Garner Color of no greater than 2, and from 28% by weight to 45% by weight of a plasticizer. The HMPSA compositions are especially useful as positioning adhesives adhering well to both cotton and microfiber.

### CROSS REFERENCE TO RELATED APPLICATIONS

This application claims the benefit of U.S. Provisional Application No. 63/202,103, filed May 27, 2021, and incorporated herein.

### BACKGROUND

This invention is directed to improved hot melt adhesive compositions for positioning. Positioning adhesives are pressure sensitive adhesive compositions, commonly hot melt pressure sensitive adhesive (HMPSA) compositions, that are used to adhere i.e., position a disposable absorbent article to a fabric or other substrate.

The variety of garments and detergents used in the world is increasing and these variables have an impact on bonding performance. Two common fabrics used today are cotton and microfiber. There is need for hot melt pressure sensitive adhesive compositions that exhibit a strong, consistent peel to both cotton and microfiber, exhibit little to no adhesive transfer to a fabric (e.g., under garment) and maintain a consistent, or even elevated peel after aging especially when exposed to body temperatures.

### SUMMARY

In one aspect, the invention features a disposable absorbent article including a topsheet, a backsheet, optionally an absorbent core disposed between the topsheet and the backsheet, a hot melt pressure-sensitive adhesive composition disposed on the garment-facing surface of the backsheet, the hot melt pressure sensitive adhesive composition including from 10% by weight to 35% by weight of a styrenic block copolymer, from 10% by weight to 60% by weight of a near white rosin ester tackifying agent having a neat Molten Garner Color of no greater than 2, and from 28% by weight to 45% by weight of a plasticizer.

In one embodiment, the disposable absorbent article is selected from the group consisting of a panty liner, a sanitary napkin, a bed liner, an incontinence pad, a disposable absorbent core, a dress shield and a nursing pad. In another embodiment, the hot melt pressure sensitive adhesive composition has a Glass Transition Temperature (Tg) of no greater than −6° C.

In another embodiment, the hot melt pressure sensitive adhesive composition has a Viscosity at 149° C. of no greater than 5,000 cP, or even a Viscosity at 121° C. of no greater than 7,000 cP. In a different embodiment, the hot melt pressure sensitive adhesive composition has a difference between the TTP Microfiber Peel and the Initial Microfiber Peel of greater than or equal to zero.

In one embodiment, the styrenic block copolymer comprises from 6% by weight to 30% by weight of a SIS block copolymer. In another embodiment, the SIS block copolymer is the styrenic block copolymer present in the highest amount. In a different embodiment, the styrenic block copolymer has an average styrene content of from 20% by weight to 35% by weight. In one embodiment, the styrenic block copolymer has an average diblock content of from 5% by weight to 50% by weight. In one embodiment, the styrenic block copolymer includes a first block copolymer that is SIS, and a second block copolymer selected from the group consisting of a second SIS and SEBS. In another embodiment, the styrenic block copolymer includes a first styrenic block copolymer that is SIS, and a second styrenic block copolymer selected from the group consisting of a second SIS, an SBS having a styrene content of no greater than 30% by weight, and a hydrogenated styrenic block copolymer.

In one embodiment, the near white rosin ester is present at from 15% by weight to 55% by weight. In a different embodiment, the near white rosin ester is present at from 20% by weight to 60% by weight. In another embodiment, the near water white rosin ester tackifying agent has a neat Molten Garner Color of no greater than 1. In one embodiment, the near water white rosin ester tackifying agent has a Ring and Ball Softening Point of from 90° C. to 120° C.

In another embodiment, the hot melt pressure sensitive adhesive composition comprises an additional tackifying agent selected from the group consisting of an aromatic modified hydrocarbon resin and an aromatic hydrocarbon resin. In a different embodiment, the hot melt pressure sensitive adhesive composition comprises an aromatic hydrocarbon resin having a Ring and Ball Softening Point of 110° C. to 130° C.

In one embodiment, the plasticizer comprises a naphthenic oil. In a different embodiment, the plasticizer comprises a naphthenic oil and a polyisobutylene. In one embodiment, the polyisobutylene has a Mw of no greater than 2000.

In one aspect, the invention features a hot melt pressure sensitive adhesive composition including from 4% by weight to 35% by weight of a first polymer comprising one or more styrene-isoprene-styrene block copolymers having an average styrene content of 12% by weight to 35% by weight and an average Melt Flow Rate (MFR) per ASTM D 1238 (200° C./5 kg) of from 5 g/10 minute to 40 g/10 minute, from 20% by weight to 60% by weight of a near white rosin ester tackifying agent having a neat Molten Garner Color of no greater than 2, and from 28% by weight to 45% by weight of a plasticizer selected from the group consisting of naphthenic oil, polyisobutylene, and combinations thereof.

In one embodiment, the hot melt pressure sensitive adhesive composition further includes from 1% by weight to 15% by weight of a second polymer selected from the group consisting of hydrogenated styrene block copolymers and styrene-butadiene-styrene block copolymers having a styrene content of less than 30% by weight.

The HMPSA compositions of this invention have strong adhesion (as tested by peel) to both cotton and microfiber. This adhesion is maintained or is even improved as the HMPSA composition is exposed to body temperatures over time. The HMPSA compositions of this invention bond especially well to microfiber. Microfiber is comprised of fine fibers of polar materials (e.g., polyester, polyamides, etc.) which are tightly woven. Due to the polarity and the tight weave, it can be difficult for generally non-polar hot melt adhesive compositions to maintain adhesion to microfiber.

Other features and advantages will be apparent from the following description of the preferred embodiments and from the claims.

### DETAILED DESCRIPTION

The HMPSA composition includes from 10% by weight to 35% by weight of a styrenic block copolymer, from 10% by weight to 60% by weight of a near white rosin ester tackifying agent having a neat Molten Garner Color of no greater than 2, and from 28% by weight to 45% by weight of a plasticizer.

The HMPSA composition can include from 6% by weight to 30% by weight of one or more styrene-isoprene-styrene block copolymers having an average styrene content of 12% by weight to 35% by weight and an average Melt Flow Rate (MFR) per ASTM D 1238 (200° C./5 kg) of from 5 g/10 minute to 40 g/10 minute, from 20% by weight to 60% by weight of a near white rosin ester tackifying agent having a neat Molten Garner Color of no greater than 2, and from 28% by weight to 45% by weight of a plasticizer selected from the group consisting of naphthenic oil, polyisobutylene, and combinations thereof.

The HMPSA composition can include from 4% by weight to 35% by weight of a first polymer comprising one or more styrene-isoprene-styrene block copolymers having an average styrene content of 12% by weight to 35% by weight and an average Melt Flow Rate (MFR) per ASTM D 1238 (200° C./5 kg) of from 5 g/10 minute to 40 g/10 minute, optionally from 1% by weight to 15% by weight of a second polymer selected from the group consisting of hydrogenated styrene block copolymers and styrene-butadiene-styrene block copolymers having a styrene content of less than 30% by weight, from 20% by weight to 60% by weight of a near white rosin ester tackifying agent having a neat Molten Garner Color of no greater than 2, and from 28% by weight to 45% by weight of a plasticizer selected from the group consisting of naphthenic oil, polyisobutylene, and combinations thereof.

The HMPSA composition is suitable for use as a positioning adhesive, which is a class of adhesive compositions that is often used to position feminine hygiene articles, such as sanitary napkins, on undergarments. These articles are removed from the undergarment after use. When the article is removed from the undergarment, preferably no adhesive composition remains on the undergarment (i.e., the undergarment is free of adhesive transfer).

The HMPSA compositions of this invention bond especially well to microfiber. It is thought that this is because the HMPSA compositions of this invention are relatively polar and further due to their flow properties (lower viscosity and low Glass Transition Temperature) can penetration better into microfiber fabric while still having the low color and odor that is expected of disposable absorbent article adhesives.

The flow properties of the HMPSA composition also enables lower application temperatures. The HMPSA composition can be applied at temperature as low at 121° C. (250° F.).

The HMPSA composition can have a difference between the TTP Microfiber Peel and the Initial Microfiber Peel of greater than or equal to zero, or even greater than zero

The HMPSA composition can have Glass Transition Temperature (Tg) of no greater than 5° C., no greater than 0° C., no greater than −6° C., no greater than −8° C., from −25° C. to 5° C., from −25° C. to 0° C., from −20° C. to −6° C., or even from −20° C. to −8° C.

The HMPSA composition can have a viscosity of no greater than 15,000 cP, no greater than 10,000 cP, from 100 cP to 15,000 cP, or even from 100 cP to 10,000 cP at 149° C.

The HMPSA composition can have a viscosity of no greater than 5,000 cP, no greater than 2,000 cP, from 100 cP to 5,000 cP, or even from 100 cP to 2,000 cP at 149° C. The HMPSA composition can have a viscosity of no greater than 7,000 cP, from 500 cP to 7,000 cP, from 2,000 cP to 7,000 cP, or even from 2,000 cP to 6,000 cP at 121° C.

Styrenic Block Copolymer

The composition includes a styrenic block copolymer.

The styrenic block copolymer has at least one A block that includes styrene and at least one B block that includes, e.g., elastomeric conjugated dienes (e.g., hydrogenated and unhydrogenated conjugated dienes), sesquiterpenes (e.g., hydrogenated and nonhydrogenated sesquiterpenes), and combinations thereof. The A blocks and the B blocks bind to one another in any manner of binding such that the resulting copolymer exhibits a variety of structures including, e.g., random, straight chained, branched, radial, star, comb, tapered, and combinations thereof. The block copolymer can exhibit any form including, e.g., linear A-B block, linear A-B-A block, linear A-(B-A) n-B multi-block, and radial (A-B) n-Y block where Y is a multivalent compound and n is an integer of at least 3, tetrablock copolymer, e.g., A-B-A-B, and pentablock copolymers having a structure of A-B-A-B-A.

Suitable styrene A blocks include, e.g., styrene, alpha-methylstyrene, o-methylstyrene, m-methylstyrene, p-methylstyrene, p-tert-butylstyrene, 2,4-dimethylstyrene, 2,4,6-trimethylstyrene, and combinations thereof.

Suitable block elastomeric conjugated diene B blocks include, e.g., butadiene (e.g., polybutadiene), isoprene (e.g., polyisoprene), 2,3-dimethyl-1,3-butadiene, 1,3-pentadiene, 1,3-hexadiene, and combinations thereof, and hydrogenated versions thereof including, e.g., ethylene, propylene, butylene and combinations thereof. Suitable B block sesquiterpenes include, e.g., beta farnesene. The styrenic block copolymer can have an unsaturated mid-block, alternately the mid-block can be saturated i.e., hydrogenated.

In addition to elastomeric conjugated diene B blocks, styrene monomer can be distributed in the mid-block.

Useful styrenic block copolymers include, e.g., poly(styrene-b-butadiene) (SB), poly(styrene-b-butadiene-b-styrene) (SBS), poly(styrene-b-isoprene) (SI), poly(styrene-b-isoprene-b-styrene) (SIS), poly(styrene-b-(ethylene-co-butylene)-b-styrene) (SEBS), poly(styrene-b-(ethylene-alt-propylene)-b-styrene) (SEPS), poly(styrene-b-isobutylene-b-styrene) (SIBS), poly(styrene-b-(ethylene-co-butylene-co-styrene)-b-styrene) (SEBSS) and combinations thereof.

In a preferred embodiment, the HMPSA composition includes at least one SIS block copolymer. In one embodiment, the SIS block copolymer is the polymer present in the highest amount. In other words, the majority of the polymer component of the HMPSA composition comprises an SIS block copolymer.

In one embodiment, the HMPSA composition does not include a SBS block copolymer having a styrene content of greater than 30% by weight, greater than 28% by weight, or even greater than 26% by weight.

In one embodiment, the HMPSA composition includes from 6% by weight to 35% by weight, from 6% by weight to 30% by weight, or even from 8% by weight to 25% by weight of a first polymer comprising one or more styrene-isoprene-styrene block copolymers having an average styrene content of from 12% by weight to 35% by weight, or even from 15% by weight to 30% by weight and an average Melt Flow Rate (MFR) per ASTM D 1238 (200° C./5 kg) of from 5 g/10 minute to 40 g/10 minute. The HMPSA composition can additionally include from 1% by weight to 15% by weight, or even from 1% by weight to 10% by weight of a second polymer selected from the group consisting of hydrogenated styrene block copolymers and styrene-butadiene-styrene block copolymers having a styrene content of no greater than 30% by weight, no greater than 28% or even no greater than 26% by weight.

The styrenic block copolymer can include more than one styrenic block copolymer. When more than one styrenic block copolymer is included the styrene content, the diblock content and the melt flow rate ranges specified below are a weight average of all the grades present.

As an example, if the HMPSA composition comprises two styrenic block copolymers A and B. Polymer A is present at 25 weight % (w_{A}) with a styrene content of 15% (s_{A}) and polymer B is present at 25 weight % (w_{B}) with a styrene content of 20 weight % (s_{B}). The average styrene content of the styrenic block copolymer is calculated in the following way:

w_{A}/(w_{A}w_{B})*s_{A}+w_{B}/(w_{A}+w_{B})*s_{B}=0.5(15)+0.5(20)=17.5 weight %.

The styrenic block copolymer can have an average styrene content of from 15% by weight to 40% by weight, from 15% by weight to 35% by weight, from 20% by weight to 35% by weight, from 20% by weight to 30% by weight, or even from 20% by weight to 28% by weight.

The styrenic block copolymer can include from 0% by weight to 50%, 5% by weight to 50% by weight, or even from 10% by weight to 40% by weight diblock.

The styrenic block copolymer can have an average Melt Flow Rate (MFR) per ASTM D 1238 (200° C./5 kg) in g/10 min of from 5 to 40, 8 to 35, or even 9 to 30.

Useful block copolymers are commercially available under the KRATON D, KRATON G, and KRATON MD series of trade designations from Kraton Corporation (Houston, Tex.) including KRATON MD 1537 H and KRATON D 1116, the VECTOR series and TAIPOL series of trade designations from Taiwan Synthetic Rubber Corporation (TSRC) (Taipei City, Taiwan) including VECTOR 4114, VECTOR 4211, TAIPOL 6151.

The HMPSA composition includes from 10% by weight to 35% by weight, from 10% by weight to 30% by weight, or even from 12% by weight to 25% by weight total styrenic block copolymer.

Near Water White Rosin Ester Tackifying Agent

The HMPSA composition of this invention includes a rosin ester tackifying agent having a near water white initial color as witnessed by a neat Molten Gardner Color of no greater than 2, or even no greater than 1. Prior art rosin ester tackifying agents having a higher neat Molten Gardner Color e.g. SYLVALITE RE 100L (near Molten Gardner Color of no greater than 4) are excluded from this category.

Examples of useful near water white rosin ester tackifying agents include e.g., glycerol esters of pale wood rosin, glycerol esters of hydrogenated rosin, glycerol esters of polymerized rosin, pentaerythritol esters of natural and modified rosins including pentaerythritol esters of pale wood rosin, pentaerythritol esters of hydrogenated rosin, pentaerythritol esters of tall oil rosin, and phenolic-modified pentaerythritol esters of rosin.

The near water white rosin ester tackifying agent can have a Ring and B all Softening Point as reported by the supplier of 90° C. to 120° C., or even from 95° C. to 115° C.

Near water white rosin ester tackifying agents include those obtained by the processes taught in U.S. Ser. No. 10/611,926B2 and US2020199408A1 which are in hereby incorporated by reference.

Useful near water white rosin ester tackifying agents include SYLVALITE 9100 which is available from Kraton Corporation (Houston, Tex.).

The HMPSA composition can include from 10% by weight to 60% by weight, from 15% by weight to 60% by weight, from 20% by weight to 60% by weight, from 30% by weight to 55% by weight, or even from 40% by weight to 55% by weight of the near water white rosin ester tackifying agent.

Additional Tackifying Agent

The HMPSA compositions of this invention can include one or more additional tackifying agents.

Suitable classes of additional tackifying agents include, e.g., aromatic, aliphatic and cycloaliphatic hydrocarbon resins, and hydrogenated versions thereof, aromatic modified aliphatic or cycloaliphatic hydrocarbon resins, and hydrogenated versions thereof; C5 tackifying agents made from monomers including dienes such as e.g. piperylene, 1,3-pentadiene, etc.; terpenes, modified terpenes and hydrogenated versions thereof; and combinations thereof. Examples of useful aliphatic and cycloaliphatic petroleum hydrocarbon resins include aliphatic and cycloaliphatic petroleum hydrocarbon resins include, e.g., branched and unbranched C9 resins and C10 resins and the hydrogenated derivatives thereof. Examples of useful polyterpene resins include hydrogenated polyterpene resins, and copolymers and terpolymers of natural terpenes (e.g., styrene-terpene, alpha-methyl styrene-terpene and vinyl toluene-terpene).

Preferred classes of tackifying agents include hydrogenated hydrocarbon resins having aromatic content. Hydrogenated hydrocarbon resins having aromatic content can be selected from the group consisting of aromatic modified hydrocarbon resin and aromatic hydrocarbon resin.

The aromatic modified hydrocarbon resins are primarily non-aromatic but include from 3% to 15% of aromatic content. The aromatic content is measured via proton Nuclear Magnetic Resonance (NMR) integration. Aromatic modified hydrocarbon resins can be selected from aliphatic and cycloaliphatic hydrocarbon resins, terpenes and modified terpenes. Useful hydrogenated aromatic modified hydrocarbon resins include ESCOREZ 5600 (9.8% aromatic content), ESCOREZ 5690 (10% aromatic content), ESCOREZ 5615 (9.9% aromatic content) and ESCOREZ 5637 (5% aromatic content).

The aromatic hydrocarbon resin is derived from aromatic vinyl monomers. Suitable examples of the aromatic hydrocarbon resins include but are not limited to aromatic hydrocarbon resins comprising monomers selected from the group consisting of styrene, alpha methyl styrene, vinyl toluene, indene, or any other aromatic monomer or end block associating monomer. Aromatic hydrocarbon resins can have greater than 40%, greater than 50%, greater than 60%, from 40% to 100%, or even from 50% by weight to 100% of aromatic content. The aromatic content is measured via proton Nuclear Magnetic Resonance (NMR) integration.

In one embodiment, the HMPSA composition includes an aromatic hydrocarbon resin having a Ring and Ball Softening Point as reported by the supplier of from 110° C. to 130° C.

Useful hydrogenated aromatic hydrocarbon resins include KRISTALEX and PLASTOLYN series of trade designations from Eastman Chemical Company (Kingsport, Tenn.) including, e.g., KRISTALEX 3100, PLASTOLYN 240 (55% aromatic content) and PLASTOLYN 290.

The HMPSA composition can include from 0% by weight to 40% by weight, from 0% by weight to 30% by weight, from 2% by weight to 30% by weight, from 5% by weight to 25%, or even from 2% by weight to 15% by weight of additional tackifying agents.

Plasticizer

The HMPSA composition includes one or more plasticizers.

The plasticizer can be a liquid at room temperature. Suitable plasticizers for use in the composition include, e.g., naphthenic oil, mineral oil, paraffin oil, synthetic liquid oligomers of polyolefins (e.g., polyisobutylene, polybutene, and polypropylene), hydrocarbon fluids, vegetable oil, functionalized versions thereof, and combinations thereof.

The plasticizer can include both an oil and polyisobutylene. The polyisobutylene preferably has a molecular weight (Mw) of <2000. The plasticizer can include both a naphthenic oil and a polyisobutylene.

Useful commercially available plasticizers include, e.g., plasticizers sold under the NYFLEX series of trade designations from Nynas Corporation (Houston, Tex.) including, e.g., NYFLEX 222B and NYFLEX 223, KAYDOL OIL from Sonneborn, LLC (Parsippany, N.J.), KRYSTOL 550 mineral oil from Petrochem Carless Limited (Surrey, England), CALSOL 5550 oil from Calumet Specialty Products Partners, LP (Indianapolis, Ind.), and TPC1160, a polyisobutylene available from TPC Group (Houston, Tex.).

The composition includes from 25% by weight to 45% by weight, from 28% by weight to 45% by weight, from 30% by weight to 45% by weight, or even from 30% by weight to 40% by weight of a plasticizer.

Optional Components

The composition can optionally include a variety of additional components including, e.g., stabilizers, antioxidants, adhesion promoters, ultraviolet light stabilizers, rheology modifiers, biocides, corrosion inhibitors, dehydrators, colorants (e.g., pigments and dyes), optical brighteners, fillers, surfactants, flame retardants, waxes, additional tackifying agents, additional polymers e.g. polar polymers e.g. EVA polymers (VA content >28%), CAPPA 6500, a polycaprolactone available from Ingevity (North Charleston, S.C.), coextrusion coatings (e.g. EPOLENE C-13 and RM6313), packaging films and combinations thereof.

Useful antioxidants include, e.g., pentaerythritol tetrakis[3,(3,5-di-tert-butyl-4-hydroxyphenyl)propionate], 2,2′-methylene bis(4-methyl-6-tert-butylphenol), phosphites including, e.g., tris-(p-nonylphenyl)-phosphite (TNPP) and bis(2,4-di-tert-butylphenyl)4,4′-diphenylene-diphosphonite, di-stearyl-3,3′-thiodipropionate (DSTDP), and combinations thereof. Useful antioxidants are commercially available under a variety of trade designations including, e.g., the IRGANOX series of trade designations including, e.g., IRGANOX 1010, IRGANOX 565, and IRGANOX 1076 hindered phenolic antioxidants and IRGAFOS 168 phosphite antioxidant, all of which are available from BASF Corporation (Florham Park, N.J.), and ETHYL 702 4,4′-methylene bis(2,6-di-tert-butylphenol), and the BNX series of trade designations including, e.g., BNX 1010 and BNX 1076 from Mayzo, Inc. (Suwanee, Ga.). When present, the adhesive composition preferably includes from about 0.1% by weight to about 2% by weight antioxidant.

Use

The hot melt pressure sensitive adhesive composition is suitable for use in a variety of articles including, e.g., disposable absorbent articles (e.g., absorbent hygiene articles), tapes (e.g., repositionable pressure sensitive adhesive tapes), and combinations thereof. Useful absorbent hygiene articles include an absorbent structure and the pressure sensitive adhesive composition disposed on a surface of the absorbent structure. The absorbent hygiene article, which is designed to be used and disposed of after a single use, can have a variety of constructions and can be suitable for use in a variety of applications including, e.g., feminine hygiene pads (e.g., panty liners and sanitary napkins), bed liners, incontinence pads, dress shields, cloth diapers or incontinence pants with disposable absorbent cores, and nursing pads. The absorbent structure of the absorbent hygiene article can include a variety of components arranged in a variety of configurations.

The pressure-sensitive adhesive composition is useful as a positioning adhesive disposed on at least one substrate surface of a disposable absorbent article and can be used to position an absorbent article on a garment such as underwear. Such disposable absorbent articles include, e.g., feminine hygiene articles such as sanitary napkins and panty liners, diapers, disposable garments having a waist opening and leg openings, and adult incontinence articles.

In one construction, the disposable absorbent article (e.g., a feminine hygiene article) includes a garment facing surface and a body facing surface, a topsheet having a garment facing surface and a body facing surface, a backsheet having a garment facing surface and a body facing surface, and an absorbent core disposed between the body facing surface of the backsheet and the garment facing surface of the topsheet.

The top sheet defines a body-facing surface of the disposable absorbent article. The absorbent core is positioned inwardly from the outer periphery of the disposable absorbent article. The absorbent structure includes a body-facing surface and a garment-facing surface positioned adjacent the backsheet. The positioning adhesive composition is disposed between a release liner and the backsheet, which is permanently adhered to the absorbent core through a permanent adhesive composition. The top sheet is permanently adhered to the absorbent core through a discontinuous permanent adhesive composition. The top sheet and the backsheet are joined together and the sealed edges of the top sheet and the backsheet define an overall sealed peripheral edge of the article. The disposable absorbent article can be of any suitable shape and size.

The top sheet is designed to contact the body of the user and is liquid permeable. The exposed surface of the liquid permeable top sheet is designed to receive aqueous fluids from the body, which fluids will then be directed away from the body of the user and toward the absorbent core. The top sheet is constructed of any suitable material that is easily penetrated by bodily exudates. The top sheet optionally includes a plurality of apertures formed therethrough to permit body fluid to pass more readily into the absorbent core.

The backsheet is liquid-impermeable and designed to face the inner surface, i.e., the crotch portion of the garment (e.g., underwear) of a user. The backsheet optionally is constructed to permit a passage of air or vapor out of the disposable absorbent article (e.g., a vapor permeable layer), while still blocking the passage of liquids.

The hot melt pressure-sensitive adhesive composition can be disposed on the garment facing surface of the adsorbent article, or even on the garment facing surface of the backsheet. A release liner optionally is disposed on the pressure-sensitive hot melt adhesive composition to protect the pressure-sensitive adhesive composition until use. The disposable absorbent article (e.g., a feminine hygiene article) optionally includes additional layers and adhesives and the components of the disposable absorbent article optionally exhibit additional functionality. Examples of additional layers, functionality and combinations thereof include dusting, wicking, acquisition, additional top sheets, multiple core layers, superabsorbent particles and compositions, wetness indicators, and combinations thereof.

Two common fabrics used today, that positioning adhesives need to bond to, are cotton and microfiber. Cotton fabric refers to weaved fabric made with 100% cotton fiber. Microfiber is a fabric created with synthetic fiber with a diameter of less than ten micrometers. Multiple materials can be used to create the microfiber fabrics, generally the materials are polar.

The composition is also useful in a variety of other applications and constructions including, e.g., forming permanent bonds, temporary bonds (e.g., removable and repositionable adhesive applications), medical dressings (e.g., wound care products), bandages, surgical pads, drapes, gowns, labels (e.g., pressure-sensitive adhesive labels), tapes (e.g., pressure-sensitive adhesive tapes), filters (e.g., pleated filters and filter frames), and combinations thereof.

The composition is useful in a variety of forms including, e.g., as a coating (e.g., continuous and discontinuous (e.g., random, pattern, array, spiral, dots, spots, and combinations thereof) coatings), film (e.g., continuous films and discontinuous films), bead, sheet, fiber, filament, web (e.g., woven and nonwoven), and combinations thereof.

The composition also can be applied to a variety of substrates including, e.g., films (e.g., polyolefin (e.g., polyethylene and polypropylene), polyester, metallized polymer, multilayer, biaxially oriented, monoaxially oriented, ethylene-vinyl acetate copolymer, polyurethane, vinyl, polyvinylidene fluoride, cellulose acetate and ethyl cellulose, and polyamide films, and combinations thereof), metal foils, release liners, porous substrates, cellulose substrates, sheets (e.g., paper and fiber sheets), paper products, woven and nonwoven webs, fibers (e.g., natural cellulose fibers such as wood pulp, cotton, silk and wool; cellulosic fibers; synthetic polymer fibers such as nylon, rayon, polyesters, acrylics, polypropylenes, polyethylene, polyvinyl chloride, and polyurethane; glass fibers; recycled fibers; and various combinations thereof), and tape backings. Useful substrates include, e.g., single layer, multilayer, treated (e.g., corona treated or chemically primed), and untreated substrates, and combinations thereof.

Various application techniques can be used to apply the adhesive composition to a substrate including, e.g., slot coating, spraying (e.g., spiral spraying and random spraying), screen printing, foaming, engraved roller, extrusion, meltblown adhesive application techniques, and combinations thereof.

The invention will now be described by way of the following examples. All parts, ratios, percentages and amounts stated in the Examples are by weight unless otherwise specified.

### EXAMPLES

### Test Procedures

Test procedures used in the examples include the following. All ratios and percentages are by weight unless otherwise indicated. The procedures are conducted at room temperature (i.e., an ambient temperature of from about 20° C. to about 25° C.) unless otherwise specified. The properties set forth for the components used in the compositions are as reported by the manufacturer unless otherwise specified.

### Viscosity Test Method

Viscosity is determined in accordance with ASTM D-3236 entitled, “Standard Test Method for Apparent viscosity of Hot Melt Adhesives and Coating Materials,” (Oct. 31, 1988), using a Brookfield Thermosel viscometer Model RVDV 2, and a number 27 spindle. The results are reported in centipoise (cP) and the test is performed at the specified temperature.

### Glass Transition Temperature (Tg) Test Method

The Tg was performed by Dynamic Mechanical Analysis (DMA) The following conditions were used: 1200-micron gap between plates, Frequency of 1 Hertz with 6% strain. The HMPSA composition was heated to 150° C. and then cooled down from 150° C. to −20° C. using a 2° C./minute ramp rate. The Tg is the temperature at which the tan delta curve exhibits a local maxima between −20° C. and 0° C.

### Test Sample Preparation Method for Peel Force to Cotton and Microfiber

A laminate is prepared by coating a sample composition onto a silicone coated release paper in a one-inch-wide pattern at an add-on weight of 20 grams per square meter (g/m²) (+/−3 g/m²) using a slot applicator and then contacting the adhesive strip with the treated side of a 1 mil (0.025 mm) thick polyethylene film to form a silicone coated release paper/adhesive/polyethylene film laminate. Test samples having a length of 4 inches (in) (10.16 cm) in the machine direction and 1.5 in (3.81 cm) in the cross-machine direction are then cut from the laminate such that the adhesive pattern is centered in the cross-machine direction of the test sample.

For cotton bonds a sheet of 124 g/m² bleached t-shirt cotton fabric (Testfabrics, Inc., West Pittston, Pa.) is cut into strips having a length of 4 in (10.16 cm) in the machine direction and a width of 1.5 in (3.81 cm) in the cross-machine, before cutting the cotton fabric, the grid work of the stitching of the fabric is examined. When the cotton fabric is stretched, the sample will exhibit greater elongation in one direction than in another direction. The cotton fabric is cut lengthwise in the direction that has less elongation. All cotton fabric strips are cut as straight as possible along the stitching grid work. If the cotton fabric strips are cut askew, an inconsistent elongation of the cotton fabric test sample will result.

The release film is removed from the adhesive and the adhesive side of each test sample is gently placed on the surface of a cotton strip such that the cotton curls up (in the lengthwise direction) toward the adhesive bond to form the composite test sample. In preparing the composite test sample, the adhesive is not pressed down onto the cotton fabric.

For the microfiber bonds a sheet of LUCERO polymeric microfiber made from 70% polyamide and 30% elastomer having a target weight of 135 g/m2 (Termileniao S. A.). Before testing the fabric is first washed without detergent and then cut into strips having a length of 8 in (20.32 cm) in the machine direction and a width of 1.5 in (3.81 cm) in the cross-machine with the long edge going along the lines of the fabric to avoid stretching.

Care is taken to cut each 8-inch strip in half creating two 4-inch-long strips. Then one of these strips is turned over to reveal the other face of the fabric. An adhesive sample is placed on each of the 4-inch strips ensuring an equal number of bonded samples are made to each face of the fabric. The two sides of the fabric exhibit different peel values, and the more difficult to bond to side of the fabric was used in peel testing experiments.

### Cotton and Microfiber Peel Force Test Method

At least five samples are prepared according to the Peel Force Sample Preparation Method. The test sample is placed on a 2-kg mechanical roll-down device and the roller is allowed to pass over the film side of the sample two times, once in the forward direction and once in the backward direction, at a rate of 305 mm/min. A timer is then activated and the sample is placed into the jaws of INSTRON-type peel tester. The polyethylene film is placed into the moving jaw, and the fabric is attached to the stationary jaw. Within one minute after the sample has been removed from the roll-down device, the sample is tested according to ASTM D1876-01 entitled, “Test Method for Determining Peel Resistance of Adhesive (T-Peel Test Method),” with the exception that the test is run at a rate of 305 mm/min, instead of 250 mm/min, over a period of ten seconds, and at least five replicates are run instead of the ten specified in ASTM D1876. The average peel force over ten seconds of peeling is recorded, and the results are reported in grams.

The initial peel force is measured 24 hours after the test sample is prepared. The two-week peel force is measured after the test sample has been subjected to accelerated aging at 50° C. for two weeks. The four-week peel force is measured after the test sample has been subjected to accelerated aging at 50° C. for four weeks.

### Time Temperature Pressure (TTP) Cotton and Microfiber Peel Force Test Method

Time, temperature and pressure peel force is determined according to the Cotton and Microfiber Peel Force Test Method set forth above with the exception that, after the test sample has been prepared and prior to testing, a 5 kilogram (kg) brass weight is placed on the test sample and the test sample is then conditioned in an oven at 40° C. for a period of time. After conditioning, the samples are removed from the oven and allowed to equilibrate to 25° C. before testing.

### Transfer

When the cotton and microfiber (or fabric) peel bonds were pulled, the surface of the fabric was evaluated for any HMPSA composition that had transferred. If transfer was seen, a yes was recorded. If no transfer was seen, a no was recorded.

### Examples

The adhesive compositions were prepared by combining and mixing the components under nitrogen in the amounts set forth in Tables 1 in a sigma blade mixer operating at 177° C.

The adhesive compositions were then tested according to at least one of the Viscosity, Glass Transition Temperature, Peel Force to Cotton, Peel Force to Microfiber and Adhesive Transfer test methods set forth above. The viscosity test method was conducted at 149° C. These results are also reported in Table 1 below.

TABLE 1

| | Control 1 | Ex 1 | Ex 2 | Ex 3 | Ex 4 | Ex 5 | Ex 6 | Ex 7 | Ex 8 | Ex 9 | Ex 10 | Ex 11 | Ex 12 |
|---|---|---|---|---|---|---|---|---|---|---|---|---|---|
| KRATON | | 2 | 4 | 4 | 4 | | | | | | | | |
| MD1537H (SEBS, | | | | | | | | | | | | | |
| 60% styrene, no | | | | | | | | | | | | | |
| diblock) | | | | | | | | | | | | | |
| VECTOR 4211 | 22 | | | | | 17 | 17 | 17 | 15 | 15 | 10 | 7 | |
| (SIS, 30% styrene, | | | | | | | | | | | | | |
| no diblock) | | | | | | | | | | | | | |
| VECTOR 4114 | | 14 | 12 | 12 | 10 | | 4 | | 6 | 2 | 7 | 10 | 7.5 |
| (SIS, 15% styrene, | | | | | | | | | | | | | |
| 42% diblock) | | | | | | | | | | | | | |
| KRATON D 1116 | | | | | | | | | | | | | 11.5 |
| (SBS, 23% styrene, | | | | | | | | | | | | | |
| no diblock) | | | | | | | | | | | | | |
| TAIPOL 6151 | | | | | | | | 4 | | 4 | 4 | 4 | |
| SEBS, 32% styrene, | | | | | | | | | | | | | |
| no diblock) | | | | | | | | | | | | | |
| Ave Styrene | 30 | 21 | 26.3 | 26.3 | 28.1 | 30 | 26.9 | 31.1 | 25.7 | 28.4 | 25.1 | 22.8 | 19.8 |
| Content | | | | | | | | | | | | | |
| (weight %) | | | | | | | | | | | | | |
| Ave Diblock | 0 | 36.8 | 31.5 | 31.5 | 30 | 0 | 8 | 0 | 12 | 4 | 14 | 20 | 16.6 |
| Content | | | | | | | | | | | | | |
| (weight %) | | | | | | | | | | | | | |
| CAPPA 6500 | | | | | 2 | 4 | | | | | | | |
| ESCOREZ 5400 | 49 | | | | | | | | | | | | |
| ESCOREZ 5615 | | 10 | 10 | | | | | | | | | | |
| PLASTOLYN 240 | | 10 | 10 | 10 | 10 | | | | | | | | |
| SYLVALITE 9100 | | 27.5 | 27.5 | 37.5 | 37.5 | 43.5 | 43.5 | 43.5 | 43.5 | 43.5 | 43.5 | 43.5 | 47.5 |
| CALSOL 5550 | 27.5 | 35 | 35 | 35 | 35 | 28 | 28 | 28 | 28 | 28 | 28 | 28 | 27 |
| TPC 1160 | | | | | | 6 | 6 | 6 | 6 | 6 | 6 | 6 | 5 |
| EPOLENE C13 | | 1 | 1 | 1 | 1 | | | | | | | | 1 |
| RM6313 | 1 | | | | | 1 | 1 | 1 | 1 | 1 | 1 | 1 | |
| Packaging Film | 0.3 | 0.25 | 0.25 | 0.25 | 0.25 | 0.25 | 0.25 | 0.25 | 0.25 | 0.25 | 0.25 | 0.25 | .25 |
| Antioxidant | 0.2 | 0.25 | 0.25 | 0.25 | 0.25 | 0.25 | 0.25 | 0.25 | 0.25 | 0.25 | 0.25 | 0.25 | .25 |
| Tg (° C.) | −5.46 | −8.88 | −10 | −8.96 | −9.06 | −16 | −18.63 | −18.29 | −17.57 | −14.98 | −15.66 | −16.18 | −14 |
| Viscosity at 160° C. | | | | | | | | | | | | | 5500 |
| (cP) | | | | | | | | | | | | | |
| Viscosity at 149° C. | 2200 | — | 1765 | 1708 | 1362 | 1470 | — | — | — | — | — | — | 8970 |
| (cP) | | | | | | | | | | | | | |
| Viscosity at 121° C. | 7588 | 4245 | 5500 | 4387 | 4200 | 4512 | 6588 | — | 6162 | — | — | — | |
| (cP) | | | | | | | | | | | | | |
| Initial Cotton Peel | 2.07 | 2.32 | 1.85 | 2.64 | 2.56 | 2.34 | 2.61 | 3.88 | 2.79 | 2.05 | 2.72 | 2.54 | 2.72 |
| (N/25 mm) | | | | | | | | | | | | | |
| TTP Cotton Peel, | 1.18 | 2.88 | 2.20 | 2.60 | 2.94 | 2.61 | NT | NT | NT | 2.49 | 4.17 | 3.15 | 4.13 |
| (N/25 mm) | | | | | | | | | | | | | |
| TTP Cotton Peel − | −0.89 | 0.56 | 0.35 | −0.04 | 0.38 | 0.27 | — | — | — | 0.44 | 1.45 | 0.61 | 1.41 |
| Initial Cotton Peel | | | | | | | | | | | | | |
| (N/25 mm) | | | | | | | | | | | | | |
| Initial Microfiber | 0.55 | 1.23 | NT | NT | 0.65 | 0.26 | 0.42 | 1.67 | 0.47 | NT | NT | NT | 1.48 |
| Peel (N/25 mm) | | | | | | | | | | | | | |
| TTP Microfiber | 0.52 | 1.26 | NT | NT | 0.91 | 0.83 | 0.83 | 4.07 | 0.88 | NT | NT | NT | 2.13 |
| Peel (N/25 mm) | | | | | | | | | | | | | |
| TTP Microfiber | −0.03 | 0.03 | — | — | 0.26 | 0.57 | 0.41 | 2.40 | 0.41 | — | — | — | .65 |
| Peel − Initial | | | | | | | | | | | | | |
| Microfiber Peel | | | | | | | | | | | | | |
| (N/25 mm) | | | | | | | | | | | | | |
| Adhesive Transfer | No | No | No | No | No | No | No | No | No | No | No | No | No |
| for any of Peels? | | | | | | | | | | | | | |

Other embodiments are within the claims.

What is claimed is:

1. A disposable absorbent article comprising:
a) a topsheet;
b) a backsheet;
c) optionally an absorbent core disposed between the topsheet and the backsheet;
d) a hot melt pressure-sensitive adhesive composition disposed on the garment-facing surface of the backsheet, the hot melt pressure sensitive adhesive composition comprising:
i) from 10% by weight to 35% by weight of a styrenic block copolymer,
ii) from 10% by weight to 60% by weight of a near white rosin ester tackifying agent having a neat Molten Garner Color of no greater than 2, and
iii) from 28% by weight to 45% by weight of a plasticizer.

2. The disposable absorbent article of claim 1 selected from the group consisting of a panty liner, a sanitary napkin, a bed liner, an incontinence pad, a disposable absorbent core, a dress shield and a nursing pad.

3. The disposable absorbent article of claim 1 wherein the hot melt pressure sensitive adhesive composition has a Glass Transition Temperature (Tg) of no greater than −6° C.

4. The disposable absorbent article of claim 1 wherein the hot melt pressure sensitive adhesive composition has a Viscosity at 149° C. of no greater than 10,000 cP.

5. The disposable absorbent article of claim 1 wherein the hot melt pressure sensitive adhesive composition has a Viscosity at 121° C. of no greater than 7,000 cP.

6. The disposable absorbent article of claim 1 wherein the hot melt pressure sensitive adhesive composition has a difference between the TTP Microfiber Peel and the Initial Microfiber Peel of greater than or equal to zero.

7. The disposable absorbent article of claim 1 wherein the styrenic block copolymer comprises from 6% by weight to 30% by weight of a SIS block copolymer.

8. The disposable absorbent article of claim 7 wherein the SIS block copolymer is the styrenic block copolymer present in the highest amount.

9. The disposable absorbent article of claim 1 wherein the styrenic block copolymer has an average styrene content of from 15% by weight to 35% by weight.

10. The absorbent article of claim 1 wherein the styrenic block copolymer comprises:
a) a first styrenic block copolymer that is SIS, and
b) a second styrenic block copolymer selected from the group consisting of a second SIS, an SBS having a styrene content of no greater than 30% by weight, and a hydrogenated styrenic block copolymer.

11. The disposable absorbent article of claim 1 wherein the near white rosin ester is present at from 15% by weight to 60% by weight.

12. The disposable absorbent article of claim 1 wherein the near water white rosin ester tackifying agent has a neat Molten Garner Color of no greater than 1.

13. The disposable absorbent article of claim 1 wherein the near water white rosin ester tackifying agent has a Ring and Ball Softening Point of from 90° C. to 120° C.

14. The disposable absorbent article of claim 1 wherein the hot melt pressure sensitive adhesive composition comprises an additional tackifying agent selected from the group consisting of an aromatic modified hydrocarbon resin and an aromatic hydrocarbon resin.

15. The disposable absorbent article of claim 1 wherein the hot melt pressure sensitive adhesive composition comprises an aromatic hydrocarbon resin having a Ring and Ball Softening Point of 110° C. to 130° C.

16. The disposable absorbent article of claim 1 wherein the plasticizer comprises a naphthenic oil.

17. The disposable absorbent article of claim 1 wherein the plasticizer comprises a naphthenic oil and a polyisobutylene.

18. The disposable absorbent article of claim 17 wherein the polyisobutylene has a Mw of no greater than 2000.

19. A hot melt pressure sensitive adhesive composition comprising:
a) from 4% by weight to 35% by weight of a first polymer comprising one or more styrene-isoprene-styrene block copolymers having an average styrene content of 12% by weight to 35% by weight and an average Melt Flow Rate (MFR) per ASTM D 1238 (200° C./5 kg) of from 5 g/10 minute to 40 g/10 minute,
b) optionally from 1% by weight to 15% by weight of a second polymer selected from the group consisting of hydrogenated styrene block copolymers and styrene-butadiene-styrene block copolymers having a styrene content of less than 30% by weight,
c) from 20% by weight to 60% by weight of a near white rosin ester tackifying agent having a neat Molten Garner Color of no greater than 2, and
d) from 28% by weight to 45% by weight of a plasticizer selected from the group consisting of naphthenic oil, polyisobutylene, and combinations thereof.

20. The hot melt pressure sensitive adhesive composition of claim 19 wherein the hot melt pressure sensitive adhesive composition comprises an additional tackifying agent selected from the group consisting of an aromatic modified hydrocarbon resin and an aromatic hydrocarbon resin.
